(19) United States
(12) Patent Application Publication
(10) Pub. No.: US 2022/0167885 A1
(43) Pub. Date: Jun. 2, 2022

(54) SYSTEMS, DEVICES, AND METHODS FOR ESTABLISHING AND/OR MAINTAINING SYNCHRONIZATION BETWEEN ENTITIES IN AN ANALYTE MONITORING ENVIRONMENT

(71) Applicant: ABBOTT DIABETES CARE INC., Alameda, CA (US)

(72) Inventors: Jean-Pierre Cole, Tracy, CA (US); Hila F. Ralston, Alameda, CA (US); Xuandong Hua, Mountain View, CA (US); Theodore J. Kunich, Pleasanton, CA (US)

(21) Appl. No.: 17/365,190

(22) Filed: Jul. 1, 2021

Related U.S. Application Data
(63) Continuation of application No. 16/022,173, filed on Jun. 28, 2018, now abandoned.
(60) Provisional application No. 62/527,024, filed on Jun. 29, 2017.

(51) Int. Cl. A61B 5/145; G16H 40/63; G16H 40/40; A61B 5/00

(52) U.S. Cl. CPC A61B 5/14532; G16H 40/63; H04L 67/12; A61B 5/7495; A61B 5/14546; G16H 40/40

(57) ABSTRACT

Embodiments for synchronous communication between devices in an analyte monitoring system are disclosed. The devices can be in on body device having an analyte sensor and a reader device. The devices can communicate with each other over one or more wireless links. Systems, devices, and methods for establishing synchronization, maintaining synchronization, and reestablishing synchronization are disclosed.

### CROSS-REFERENCE TO RELATED APPLICATIONS

This application is a continuation of U.S. patent application Ser. No. 16/022,173, filed Jun. 28, 2018, which claims priority to and the benefit of U.S. Provisional Application No. 62/527,024, filed Jun. 29, 2017, both of which are incorporated by reference herein in their entireties for all purposes.

### FIELD

The subject matter described herein relates generally to systems, devices, and methods for establishing and/or maintaining synchronization in wireless radio frequency (RF) communications between electrical devices, such as devices operating within an analyte monitoring environment.

### BACKGROUND

The detection and/or monitoring of analyte levels, such as glucose, ketones, lactate, oxygen, hemoglobin A1C, or the like, can be vitally important to the health of an individual having diabetes. Diabetics generally monitor their glucose levels to ensure that they are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including convenience, testing discretion, pain associated with glucose testing, and cost.

For these and other reasons, needs exist for improved analyte monitoring systems, devices, and methods.

### SUMMARY

Provided herein are example embodiments of systems, devices, and methods for improved wireless communication. These embodiments are particularly suited for use within and analyte monitoring system having an on body device and a reader device where the reader device intermittently collects information transmitted from the on body device. In many embodiments, the on body device and reader device are configured to communicate over to different wireless links, one of which is a synchronous link for the purpose of maintaining continuous communication. Various information, such as alarm notifications, can be transmitted over the synchronous link. Example embodiments provide for maintaining the synchronous link with expenditure of minimal power by the reader device. Some of these embodiments entail the use of a timing calibration value to adjust the timing of transmission sent over the synchronous link based upon actual performance characteristics of the on body device and/or environmental factors. Example embodiments are provided for establishing and/or reestablishing synchronous communication over this synchronous link, in some cases by use of the second wireless link.

Other systems, devices, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is an illustrative view depicting an example embodiment of an in vivo analyte monitoring system.

FIG. 2 is a block diagram of an example embodiment of a reader device.

FIG. 3 is a block diagram depicting an example embodiment of an on body device.

FIGS. 4A and 4B are timing diagrams depicting examples of synchronous communication within in analyte monitoring system.

FIGS. 4C and 4D our timing diagrams depicting examples of drift within a synchronous communication system.

FIG. 5 is a flow diagram depicting an example embodiment of determining a timing calibration value.

FIG. 6A is a flow diagram depicting an example embodiment of a method of using a timing calibration value to determine appropriate times of communication.

FIG. 6B is a timing diagram depicting an example of synchronous communication using the method of FIG. 6A.

FIG. 7 is a flow diagram depicting another example embodiment of a method of using a timing calibration value to determine appropriate times of communication.

FIGS. 8A and 8B are timing diagrams depicting example embodiments of synchronous communication by monitoring communication intervals.

FIG. 8C is a flow diagram depicting an example embodiment of a method of synchronous communication by monitoring communication intervals.

FIGS. 9A and 9B are flow diagrams depicting example embodiments of methods of reestablishing synchronous communication.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

Generally, embodiments of the present disclosure are used with systems, devices, and methods for detecting at least one analyte, such as glucose, in a bodily fluid (e.g., subcutaneously within the interstitial fluid (“ISF”) or blood, within the dermal fluid of the dermal layer, or otherwise). Accordingly, many embodiments include in vivo analyte sensors structurally configured so that at least a portion of the sensor is, or can be, positioned in the body of a user to obtain information about at least one analyte of the body. However, the embodiments disclosed herein can be used with in vivo analyte monitoring systems that incorporate in vitro capability, as well as purely in vitro or ex vivo analyte monitoring systems, including those systems that are entirely non-invasive.

Furthermore, the embodiments described herein can be used with devices that sense biometrics other than analyte data, such as heart rate, blood pressure, body temperature, perspiration, intraocular pressure, and others. The embodiments described herein can be used with devices that sense movement and/or activity level alone or in combination with any other metric. The embodiments described herein are thus not limited to medical applications and can be used with other, non-medical systems, where RF communication between devices is employed.

Before describing the embodiments in detail, however, it is first desirable to describe examples of devices that can be present within, for example, an in vivo analyte monitoring system, as well as examples of their operation, all of which can be used with the embodiments described herein.

### Example Embodiments of Analyte Monitoring Systems

In vivo monitoring systems can include a sensor that, while positioned in vivo, makes contact with the bodily fluid of the user and senses the analyte levels contained therein. The sensor can be part of a OBD that resides on the body of the user and contains the electronics and power supply that enable and control the analyte sensing. The on body device, and variations thereof, can also be referred to as a “sensor device,” an “on-body electronics device,” a “sensor control device,” or a “sensor communication device,” to name a few. As used herein, these terms are not limited to devices with in vivo analyte sensors, and encompass devices that have ex vivo sensors of other types, whether biometric (e.g., photonic analyte sensors, heart rate sensors, temperature sensors, etc.) or non-biometric. The term “on body” encompasses devices that reside directly on the body (e.g., attached to the skin), are wholly within the body (e.g., a fully implanted device), or are in close proximity to the body, such as a wearable device (e.g., glasses, watch, wristband or bracelet, neckband or necklace, etc.) or a device in a pocket, etc.

In vivo monitoring systems can also include one or more reader devices that read information about a sensed level from the on body device. These reader devices can process and/or display the sensed analyte information, in any number of forms, to the user. These devices, and variations thereof, can be referred to as “handheld reader devices,” “readers,” “handheld electronics” (or handhelds), “portable data processing” devices or units, “information receivers,” “receiver” devices or units (or simply receivers), “relay” devices or units, or “remote” devices or units, to name a few.

In vivo analyte monitoring systems can be differentiated from “in vitro” systems that contact a biological sample outside of the body, and “ex vivo” systems that gain information about the body or a substance within the body but that do so while remaining wholly outside the body without extracting a biological sample from inside the body. In vitro systems can include a meter device that has a port for receiving an analyte test strip carrying a bodily fluid of the user, which can be analyzed to determine the user's analyte level. As mentioned, the embodiments described herein can be used with in vivo systems, ex vivo systems, in vitro systems, and combinations thereof.

The embodiments described herein can be used to monitor and/or process information regarding any number of one or more different analytes. Analytes that may be monitored include, but are not limited to, acetyl choline, amylase, bilirubin, cholesterol, chorionic gonadotropin, glycosylated hemoglobin (HbAlc), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

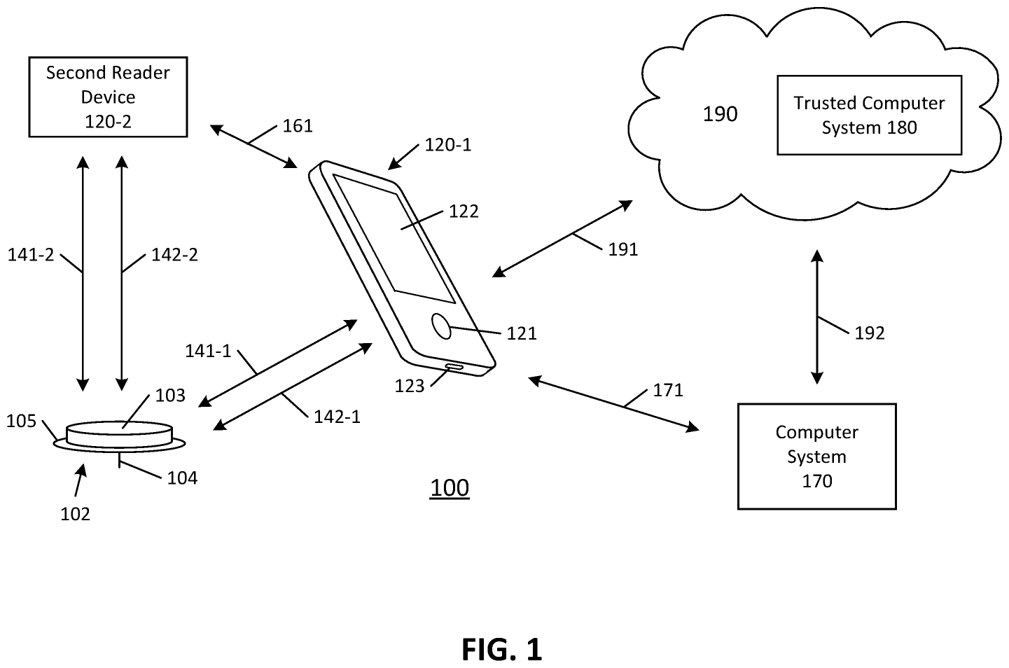

Example embodiments of in vivo analyte monitoring systems can include one or more on body devices, one or more reader devices, and one or more computer systems capable of communicating in a highly interconnected fashion. FIG. 1 is an illustrative and block diagram depicting an example embodiment of an in vivo analyte monitoring system 100 having an on body device (OBD) 102, a first reader device 120-1, a second reader device 120-2, a local or remote computer system 170, and a trusted computer system 180 (e.g., a server), each of which can be configured to communicate over a communications network 190. References to reader device 120 herein refer to both reader device 120-1 and 120-2.

OBD 102 can communicate with reader device 120 over two or more wireless communication paths, links, or channels 141 and 142, which can be uni-directional or bi-directional. Links 141 and 142 are formed by communication circuitry and antennae present in OBD 102 and reader device 120. In some embodiments, the capability for devices 102 and 120 to communicate over an additional wired communication path, such as a universal serial bus (USB) cable (not shown), can be implemented.

Wireless communication link 141 can have various implementations. In some embodiments, communication link 141 uses near field electromagnetic induction to communicate. Such links are sometimes referred to as close proximity communications as they require the transmitting and receiving devices to be in relatively close proximity as compared to far field (or transition zone) communications. Communication using electromagnetic induction generally occurs within a two wavelength distance, more typically within one wavelength distance between the transmitting and receiving devices. In many embodiments, electromagnetic induction-based communications occur only within one foot (12 inches) of range or shorter, dependent upon frequency and power of transmission, among others. Examples include “Near Field Communication” (NFC) protocols, which refer to a number of protocols (or standards) that set forth operating parameters, modulation schemes, coding, transfer speeds, frame format, and command definitions for NFC devices. Some examples of NFC devices operate at 13.56 Megahertz (Mhz). The following is a non-exhaustive list of examples of these protocols: ECMA-340, ECMA-352, ISO/IEC 14443, ISO/IEC 15693, ISO/IEC 18000-3, ISO/IEC 18092, and ISO/IEC 21481, all of which are incorporated by reference herein in their entirety and for all purposes. Examples also include Radio Frequency Identification (RFID) protocols.

Responsive communications using electromagnetic induction can be passively generated, where power conveyed by a transmission from a first device is captured by the receiving second device and used to power transmission of a response by the second device back to the first device. Responsive communications using electromagnetic induction can be actively generated, such that the receiving second device uses power from its own power source alone, or in combination with power captured from the received transmission, to power transmission of the responsive communication back to the first device.

The sending of a transmission, e.g., a request for analyte data, from reader device 120 to OBD 102 can cause OBD 102 to respond with a transmission of its own, e.g., analyte data obtained or derived from a measurement made by sensor 104. This process of transmitting from reader 120 to OBD 102 and receiving a response from OBD 102 can be referred to as “scanning” or conducting a “scan” of OBD 102. In many embodiments, OBD 102 is configured as a passive device where the power from a transmission from reader device 120 received over link 141 is captured and used to power transmission of the responsive communication from OBD 102 back to reader device 120. This can be referred to as a “passive scan.” In such embodiments, OBD 102 can power transmissions without using power from a power source (e.g., a coin cell battery) internal to OBD 102. In other embodiments, OBD 102 can be configured as an active device where the power from a transmission from reader device 120 received over link 141 is or is not captured, and the power used to transmit the responsive communication from OBD 102 back to reader device 120 is generated entirely or in part by the power source internal to OBD 102. This can be referred to as an “active scan.”

Wireless communication link 142 utilizes communication protocols other than those used by link 141 and relies primarily on the longer range far field characteristics of electromagnetic transmission, where transmission does not occur only through inductive coupling. Link 142 can communicate over the same close proximity communication range as link 141 and substantially farther. Link 142 can also have various implementations. To form link 142, OBD 102 and reader 120 can include communication circuitry and antennae configured to communicate over standardized or proprietary protocols and formats. For example, link 142 can be formed in an ultra-high frequency (UHF) band (for example, between 450-470 Megahertz). Link 142 can also be formed in other frequency bands and using other protocols, such as a Bluetooth or Bluetooth Low Energy protocol, Wi-Fi protocol, other proprietary protocol, or the like, including those communication protocols in existence as of the date of this filing or their later developed variants. In many embodiments described herein, link 142 utilizes a synchronous scheme without polling, e.g., where both the transmitting device and receiving device each maintain a clock or oscillator and seek to transmit and receive at periodic intervals derived from their respective clocks or oscillators, without the use of periodic polling or timing transmission. The transmission of periodic polling or timing signals (and corresponding monitoring for receipt of such signals) can result in higher power usage, which in turn can require a larger and more costly power supply (e.g., battery) or can cause shorter battery life. While both links 141 and 142 utilize RF transmissions, for ease of differentiation they will be referred to herein as near field link 141 and RF link 142.

OBD 102 can be configured to communicate with multiple reader devices 120, and this is shown in FIG. 1 by the presence of first reader device 120-1 capable of communication with OBD 102 over near field link 141-1 and far field link 142-1, and by the presence of second reader device 120-2 capable of communication with OBD 102 over near field link 141-2 and far field link 142-2. Additional reader devices 120 can also be present.

Reader device 120 can communicate with multiple OBDs 102. For example, each reader device 120 can communicate with a first OBD 102 on the body of a user over the first OBD's operating lifetime, and then that OBD 102 can be discarded and replaced with a second OBD 102 on the body of the user, which the same reader device 120 can again communicate with. In some embodiments, a particular reader device 120 can communicate with multiple OBDs concurrently, located on the same or different users.

Reader device 120 is also capable of wired, wireless, or combined communication with other devices. FIG. 1 depicts reader device 120-1 in communication with computer system 170 (e.g., a local or remote computer system) over communication link, path, or channel 171, and also in communication with a network 190, such as the internet or the cloud, over communication link, path, or channel 191. (Reader device 120-2 can also communicate with these devices in like fashion, but those connections are not shown for ease of illustration.) Reader device 120-1 can communicate with a trusted computer system 180 by way of network 190 using link 191. Trusted computer system 180 can communicate with computer system 170 over communication link, path, or channel 192. For example, trusted computer system 180 can be a server that serves analyte analytics software to reader device 120-1 and/or computer system 170, for example, in the form of a downloadable software application or “app” or as an internet browser accessible web page.

Communication links 171, 191, and 192 can be wireless, wired, or both, can be uni-directional or bi-directional, and can be part of a telecommunications network, such as a Wi-Fi network, a local area network (LAN), a wide area network (WAN), the internet, or other data network. In some cases, communication paths 171 and 172 can be, at least in part, the same path (e.g., such as when communicating over Wi-Fi). All communications over the various paths can be encrypted and OBD 102, reader device 120-1, reader device 120-2, computer system 170, and trusted computer system 180 can each be configured to encrypt and decrypt those communications sent and received.

Variants of devices 102 and 120, as well as other components of an in vivo-based analyte monitoring system that are suitable for use with the system, device, and method embodiments set forth herein, are described in U.S. Publication No. 2011/0213225 (the '225 Publication), which is incorporated by reference herein in its entirety for all purposes.

Referring again to FIG. 1, OBD 102 can include a housing 103 containing analyte monitoring circuitry and a power source. In this embodiment, the analyte monitoring circuitry is electrically coupled with an analyte sensor 104 that extends through an adhesive patch 105 and projects away from housing 103. Adhesive patch 105 contains at least one adhesive layer (not shown) for attachment to a skin surface of the body of the user, and optionally a second adhesive layer on the opposite surface for attachment to housing 103. Other forms of attachment to the body and/or housing 103 may be used, in addition to or instead of adhesive.

Analyte sensor 104 is adapted to be at least partially inserted into the body of the user, where it can make fluid contact with that user's bodily fluid (e.g., ISF, dermal fluid, or blood) and be used, along with the analyte monitoring circuitry, to measure analyte-related data of the user. Sensor 104 and any accompanying sensor electronics can be applied to the body in any desired manner. For example, an insertion device (not shown) can be used to position all or a portion of analyte sensor 104 through an external surface of the user's skin and into contact with the user's bodily fluid. In doing so, the insertion device can also position OBD 102 with adhesive patch 105 onto the skin. In other embodiments, the insertion device can position sensor 104 first, and then accompanying electronics (e.g., wireless transmission circuitry and/or data processing circuitry, and the like) can be coupled with sensor 104 afterwards, either manually or with the aid of a mechanical device. Examples of insertion devices are described in U.S. Publication Nos. 2008/0009692, 2011/0319729, 2015/0018639, 2015/0025345, and 2015/0173661, all which are incorporated by reference herein in their entireties and for all purposes.

After collecting raw analog data from the user's body, OBD 102 can optionally apply analog signal conditioning to the data and convert the analog data into a digital form of the conditioned raw data. In some embodiments, this digital raw data can be encoded for transmission to another device, e.g., reader device 120, which then algorithmically processes that digital raw data into a final form representative of the user's measured biometric (e.g., a form readily made suitable for display to the user). This algorithmically processed data can then be formatted or graphically processed for digital display to the user. In other embodiments, OBD 102 itself can algorithmically process the digital raw data into the final form that is representative of the user's measured biometric (e.g., analyte level) and then encode and wirelessly communicate that data to reader device 120, which in turn can format or graphically process the received data for digital display to the user. In other embodiments, OBD 102 can graphically process the final form of the data such that it is ready for display, and display that data on a display of OBD 102 or transmit the data to reader device 120. In some embodiments, the final form of the biometric data (prior to graphic processing) is used by the system (e.g., incorporated into a diabetes monitoring regime) without processing for display to the user. In some embodiments, OBD 102 and reader device 120 transmit the digital raw data to another computer system for algorithmic processing and display. The transmissions of these various forms of data can occur over either or both of links 141 and 142.

Each reader device 120 within system 100 can include a display 122 to output information to the user and/or to accept an input from the user, and an optional input component 121 (or more), such as a button, actuator, touch sensitive switch, capacitive switch, pressure sensitive switch, jog wheel or the like, to input data, commands, or otherwise control the operation of reader device 120. In certain embodiments, display 122 and input component 121 may be integrated into a single component, for example, where the display can detect the presence and location of a physical contact touch upon the display, such as a touch screen user interface. In certain embodiments, input component 121 of reader device 120 may include a microphone and reader device 120 may include software configured to analyze audio input received from the microphone, such that functions and operation of the reader device 120 may be controlled by voice commands. In certain embodiments, an output component of reader device 120 includes a speaker (not shown) for outputting information as audible signals. Similar voice responsive components such as a speaker, microphone and software routines to generate, process and store voice driven signals may be included in OBD 102.

Reader device 120 can also include one or more data communication ports 123 for wired data communication with external devices such as computer system 170 or OBD 102. Example data communication ports include all types of serial or parallel connectors, including all variants of USB ports, RS-232 ports, Ethernet ports, Firewire ports, or other similar data communication ports configured to connect to the compatible data cables. Reader device 120 may also include an integrated or attachable in vitro glucose meter, including an in vitro test strip port (not shown) to receive an in vitro glucose test strip for performing in vitro blood glucose measurements.

Reader device 120 can display the measured biometric data wirelessly received from OBD 102 and can also be configured to output alarms, alert notifications, glucose values, etc., which may be visual, audible, tactile, or any combination thereof. Further details and other display embodiments can be found in, e.g., U.S. Publication No. 2011/0193704, which is incorporated herein by reference in its entirety for all purposes.

Reader device 120 can function as a data conduit or relay to transfer the measured data from OBD 102 to computer system 170 or trusted computer system 180. In certain embodiments, the data received from OBD 102 may be stored (permanently or temporarily) in one or more memories of reader device 120 prior to uploading to system 170, 180 or network 190.

Computer system 170 may be a personal computer, a server terminal, a laptop computer, a tablet, or other suitable data processing device. Computer system 170 can be (or include) software for data management and analysis and communication with the components in analyte monitoring system 100. Computer system 170 can be used by the user or a medical professional to display and/or analyze the biometric data measured by OBD 102. In some embodiments, OBD 102 can communicate the biometric data directly to computer system 170 without an intermediary such as reader device 120, or indirectly using an internet connection (also optionally without first sending to reader device 120). Operation and use of computer system 170 is further described in the '225 Publication incorporated herein. Analyte monitoring system 100 can also be configured to operate with a data processing module (not shown), also as described in the incorporated '225 Publication.

Trusted computer system 180 can be within the possession of the manufacturer or distributor of OBD 102, either physically or virtually through a secured connection, and can be used to perform authentication of OBD 102, for secure storage of the user's biometric data, and/or as a server that serves a data analytics program (e.g., accessible via a web browser) for performing analysis on the user's measured data.

### Example Embodiments of Reader Devices

Reader device 120 can be a dedicated reader device that is custom manufactured for the purpose of interfacing with OBD 102. Reader device 120 can also be a mobile communication device such as a mobile telephone including, but not limited to, a Wi-Fi or internet enabled smart phone, tablet, or personal digital assistant (PDA). Reader device 120 can also be configured as a mobile smart wearable electronics assembly, such as a smart glass or smart glasses, or a smart watch or wristband.

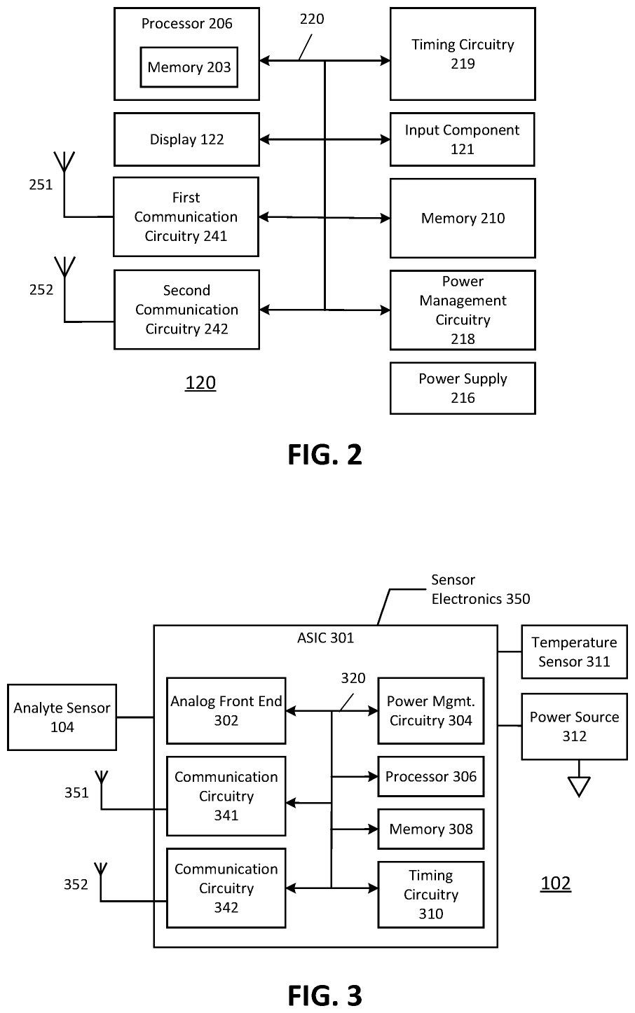

FIG. 2 is a block diagram of an example embodiment of a reader device 120 configured as a dedicated reader. Here, reader device 120 includes input component 121, display 122, and processor or processing circuitry 206 with memory 203, first communication circuitry 241 coupled with a first antenna 251, second communication circuitry 242 coupled with a second antenna 252, a memory 210, a power supply 216, power management circuitry 218, and a clock or timing circuitry 219.

Dedicated reader 120 can be implemented in a highly interconnected fashion, where power supply 216 is coupled with each component shown in FIG. 2 and where those components that communicate or receive data, information, or commands (e.g., processor 206, memory 203, memory 210, power management circuitry 218, timing circuitry 219, input component 121, display 122, first communication circuitry 241, and second communication circuitry 242), can be communicatively coupled with every other such component over, for example, one or more communication connections or buses 220. FIG. 2 is an abbreviated representation of the typical hardware and functionality that resides within a dedicated reader and those of ordinary skill in the art will readily recognize that other hardware and functionality (e.g., codecs, drivers, glue logic) can also be included.

First communication circuitry 241 and antenna 251 are configured for communication (transmission and/or reception) over communication link 141, and second communication circuitry 242 and antenna 252 are configured for communication over communication link 142. In some embodiments, antenna 251 and antenna 252 can be a single shared antenna (e.g., capable of transmission and reception over NFC and UHF frequencies). Communication circuitry 241 and 242 can be implemented as one or more chips and/or components (e.g., transmitter, receiver, transceiver, encoder, decoder, and/or other communication circuitry) that perform the functions for communications over the respective communications links 141 and 142. Communication circuitry 241 and 242 can receive timing information from timing circuitry 219. Timing circuitry 310 can include a crystal oscillator, phase-locked loop (PLL), and/or other circuitry for generating a stable frequency for timing purposes.

Antennas 251 and 252 can be configured according to the needs of the application and communication protocol. Antennas 251 and 252 can have the same or different configuration and can be, for example, a printed circuit board (PCB) trace antenna, a ceramic antenna, or a discrete metallic antenna. Antennas 251 and 252 can be configured as a monopole antenna, a dipole antenna, an F-type antenna, a loop antenna, and others.

Processor 206 can include one or more processors, microprocessors, controllers, and/or microcontrollers, each of which can be a discrete chip or distributed amongst (and a portion of) a number of different chips. Here, processor 206 includes on-board memory 203. Processor 206 can interface with communication circuitry 241 and 242 and perform analog-to-digital conversions, encoding and decoding, digital signal processing and other functions that facilitate the conversion of data signals into a format (e.g., in-phase and quadrature) suitable for provision to communication circuitry 241 and 242, which can then transmit the signals wirelessly. Processor 206 can also interface with communication circuitry 241 and 242 to perform the reverse functions necessary to receive a wireless transmission and convert it into digital data or information.

Processor 206 can execute software instructions stored in memory 203 or 210. These instructions can cause processor 206 to cause communication circuitry 241 and 242 to transmit, can cause processor 206 to read and act on received transmissions, can cause processor 206 to read input from input component 121, to display data or information on display 122, to read input from display 122 when implemented as a touchscreen for example, to adjust the timing of timing circuitry 219, to process data or information received from other devices (e.g., analyte data, calibration information, synchronization information received from OBD 102, and others), to perform tasks to maintain synchronization with OBD 102, and others.

Memory 210 can be shared by one or more of the various functional units present within reader device 120, or can be distributed amongst two or more of them (e.g., as separate memories present within different chips). Memory 210 can also be a separate chip of its own. Memories 203 and 210 are non-transitory, and can be volatile (e.g., RAM, etc.) and/or non-volatile memory (e.g., ROM, flash memory, F-RAM, etc.).

Power supply 216 can include one or more batteries, which can be rechargeable or single-use disposable batteries. Power management circuitry 218 can regulate battery charging and monitor usage of power supply 216, boost power, perform DC conversions, and the like.

Reader device 120 can also include or be integrated with a drug (e.g., insulin, etc.) delivery device such that they, e.g., share a common housing. Examples of such drug delivery devices can include medication pumps having a cannula that remains in the body to allow infusion over a multi-hour or multi-day period (e.g., wearable pumps for the delivery of basal and bolus insulin). Reader device 120, when combined with a medication pump, can include a reservoir to store the drug, a pump connectable to transfer tubing, and an infusion cannula. The pump can force the drug from the reservoir, through the tubing and into the diabetic's body by way of the cannula inserted therein. Other examples of drug delivery devices that can be included with (or integrated with) reader device 120 include portable injection devices that pierce the skin only for each delivery and are subsequently removed (e.g., insulin pens). A reader device 120, when combined with a portable injection device, can include an injection needle, a cartridge for carrying the drug, an interface for controlling the amount of drug to be delivered, and an actuator to cause injection to occur. The device can be used repeatedly until the drug is exhausted, at which point the combined device can be discarded, or the cartridge can be replaced with a new one, at which point the combined device can be reused repeatedly. The needle can be replaced after each injection.

The combined device can function as part of a closed-loop system (e.g., an artificial pancreas system requiring no user intervention to operate), semi-closed loop system (e.g., an insulin loop system requiring seldom user intervention to operate, such as to confirm changes in dose), or an open loop system. For example, the diabetic's analyte level can be monitored in a repeated automatic fashion by OBD 102, which can then communicate that monitored analyte level to reader device 120, and the appropriate drug dosage to control the diabetic's analyte level can be automatically determined and subsequently delivered to the diabetic's body. Software instructions for controlling the pump and the amount of insulin delivered can be stored in memory 203 and/or 210 of reader device 120 and executed by processing circuitry 206. These instructions can also cause calculation of drug delivery amounts and durations (e.g., a bolus infusion and/or a basal infusion profile) based on the analyte level measurements obtained directly or indirectly from OBD 102. In some embodiments OBD 102 can determine the drug dosage and communicate that to reader device 120.

### Example Embodiments of on Body Devices

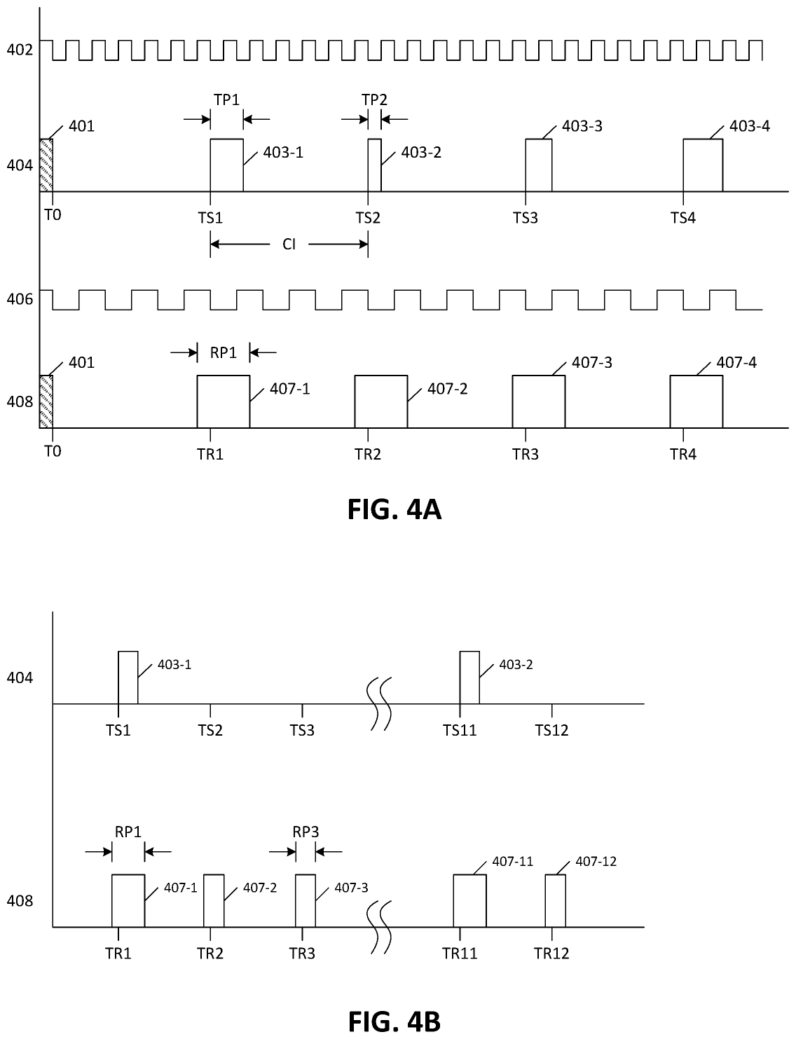

FIG. 3 is a block diagram depicting an example embodiment of OBD 102 having analyte sensor 104 and sensor electronics 350 (including analyte monitoring circuitry). Sensor electronics can be implemented in one or more semiconductor chips. In the embodiment of FIG. 3, sensor electronics 350 are in a single semiconductor chip 351 that can be a custom application specific integrated circuit (ASIC). Shown within ASIC 351 are certain high-level functional units, including an analog front end (AFE) 302, power management (or control) circuitry 304, processor or processing circuitry 306, memory 308, timing circuitry 310, first communication circuitry 341 and second communication circuitry 342. In this embodiment, both AFE 302 and processor 306 are used as analyte monitoring circuitry, but in other embodiments either circuit (or others) can perform the analyte monitoring function.

OBD 102 can be implemented in a highly interconnected fashion, where power supply 312 is coupled with each component shown in FIG. 3 and where those components that communicate or receive data, information, or commands (e.g., AFE 302, power management circuitry 304, processor 306, memory 308, timing circuitry 310, first communication circuitry 341, and second communication circuitry 342), can be communicatively coupled with every other such component over, for example, one or more communication connections or buses 320. FIG. 3 is an abbreviated representation of the typical hardware and functionality that resides within a dedicated reader and those of ordinary skill in the art will readily recognize that other hardware and functionality (e.g., codecs, drivers, glue logic) can also be included.

Communication circuitry 341 and 342 can be coupled to antennas 351 and 352, respectively, which can be on chip or off chip (as shown here). First communication circuitry 341 and antenna 351 are configured for communication (transmission and/or reception) over communication link 141, and second communication circuitry 342 and antenna 352 are configured for communication over communication link 142. In some embodiments, antenna 351 and antenna 352 can be a single shared antenna (e.g., capable of transmission and reception over NFC and UHF frequencies). Communication circuitry 341 and 342 can be implemented as one or more components (e.g., transmitter, receiver, transceiver, passive circuit, encoder, decoder, and/or other communication circuitry) that perform the functions for communications over the respective communications links 141 and 142. Communication circuitry 341 and 342 can receive timing information from timing circuitry 310. Timing circuitry 310 can include a crystal oscillator, phase-locked loop (PLL), and/or other circuitry for generating a stable frequency for timing purposes.

Although not limited to such, in some embodiments, communication circuitry 341 is passive and only uses power harvested from a transmission received from a second device (e.g., reader 120) to generate and propagate a response transmission back to the second device (such as when link 141 is an NFC link). In these and other embodiments, communication circuitry 342 can be active and can use power from OBD power source 312 to generate and propagate a transmission to a second device. The active communication circuitry 342 permits OBD 102 to generate a transmission spontaneously and with prompting from another device (e.g., without first receiving a request, polling signal, timing signal, and the like from the second device).

Processor 306 can include one or more processors, microprocessors, controllers, and/or microcontrollers, each of which can be a discrete chip or distributed amongst (and a portion of) a number of different chips. Processor 306 can interface with communication circuitry 341 and 342 and perform analog-to-digital conversions, encoding and decoding, digital signal processing and other functions that facilitate the conversion of data signals into a format (e.g., in-phase and quadrature) suitable for provision to communication circuitry 341 and 342, which can then transmit the signals wirelessly. Processor 306 can also interface with communication circuitry 341 and 342 to perform the reverse functions necessary to receive a wireless transmission and convert it into digital data or information.

Processor 306 can execute software instructions stored in memory 308. These instructions can cause processor 306 to cause communication circuitry 341 and 342 to transmit a communication generated by processor 306, can cause processor 306 to read and act on received transmissions, to adjust the timing of timing circuitry 310, to collect temperature information from a temperature sensor, to record and/or process a measurement from analyte sensor 104, to monitor collected analyte data for actual or potential alarm conditions, to generate and cause the transmission of an alarm indication using communication circuitry 342, to process data or information received from other devices (e.g., reader 120), to perform tasks to maintain synchronization with reader 120, and others.

Memory 308 is also included within ASIC 301 and can be shared by the various components present within ASIC 301, or can be distributed amongst two or more of them. Memory 308 can also be a separate chip. Memory 308 is non-transitory and can be volatile and/or non-volatile memory. In this embodiment, ASIC 301 is coupled with an optional temperature (or other environmental factor) sensor 311 and power source 312, which can be a coin cell battery, or the like. AFE 302 interfaces with in vivo analyte sensor 104 and receives measurement data therefrom, converts to digital form and outputs to processor 306 which in turn can, in some embodiments, process in any of the manners described elsewhere herein. This data can then be provided to communication circuitry 341 and 342 for sending, by way of antennas 351 and 352, to reader device 120 (not shown), for example, where minimal further processing is needed by the resident software application to display the data. Antennas 351 and 352 can be configured according to the needs of the application and communication protocol. Antennas 351 and 352 can have the same or different configuration and can be, for example, a printed circuit board (PCB) trace antenna, a ceramic antenna, or a discrete metallic antenna. Antennas 351 and 352 can be configured as a monopole antenna, a dipole antenna, an F-type antenna, a loop antenna, and others.

### Example Embodiments of Communication Over Near Field and RF Links

In many embodiments, the user can obtain a current analyte reading by bringing reader 120 in close proximity with OBD 102 and scanning OBD 102 with reader 120. A scan can be initiated by the user inputting a scan request into reader 120 (e.g., such as by pressing a selectable “Scan” field on a touchscreen display 122). Upon receiving the request, reader 120 can display on-screen instructions for the user to bring reader 120 into close proximity with OBD 102. During this time (and for a predetermined period of up to, for example, 15 or 30 seconds), reader 120 continually sends requests for analyte data to OBD 102 over near field link 141. Reader 120 can continue to transmit until receiving a response from OBD 102. The response from OBD 102 can include the requested analyte data, and is also communicated over near field link 141. Reader 120 can then process the received analyte data as needed and display it to the user on display 122. Other information and commands can also be exchanged by scanning over near field link 141. For example, reader 120 can cause initial activation of OBD 102 through a scan. OBD 102 can also communicate additional information and data in response to a scan, such as historical analyte data (e.g., data collected at various times since the last scan, or all of the analyte data collected over a previous time range, such as 8 hours, 16 hours, 24 hours, etc.). This historical analyte data can be displayed along with the current analyte reading.

In many embodiments, OBD 102 (e.g., processor 306) can monitor analyte measurements collected with analyte sensor 104 and detect whether one or more of those measurements violate, satisfy, or otherwise meet an alarm condition. This monitoring process can occur immediately upon the collection or sampling of a new measurement, or can occur at periodic intervals (e.g., every one minute, every five minutes, every ten minutes, etc.) independent of how many measurements have been collected or sampled in the time period. If an alarm condition is met, then an indication of the alarm can be transmitted from OBD 102 to reader 120 over RF link 142. System 100 utilizes RF link 142 because it is a longer range link than near field link 141, and thus is more likely to reach reader 120. Also, communication circuitry 341 for near field link 141 may be passive, and thus cannot spontaneously transmit the alarm indication without first receiving a transmitted request from which to harvest power. Further, the longer range nature of link 142 facilitates the maintenance of link 142 in continuous fashion so long as both devices are within range of each other.

Various conditions can signal an alarm. The conditions can reflect an actual or predicted physiological condition that poses an elevated risk or threat to the user's health, and which may require intervention by a medical professional. Some examples of such alarms and conditions include: a low analyte alarm (e.g., a hypoglycemia alarm) where a measurement has a lower value than a threshold, a high analyte alarm (e.g., a hyperglycemia alarm) where a measurement has a higher value than a threshold, a rapid rise alarm where multiple measurements exhibit an increase in analyte level that exceeds a threshold rate, a rapid fall alarm where multiple measurements exhibit a decrease in analyte level that exceeds a threshold rate, a predicted low analyte alarm (e.g., an impending hypoglycemic condition) where multiple measurements indicate a decrease in analyte level at a rate that will result in violation of an actual low analyte threshold (this alarm can also require a recent measurement to be less than a predicted low analyte threshold that indicates proximity to the actual low analyte threshold), and a predicted high analyte alarm (e.g., an impending hyperglycemic condition) where multiple measurements indicate an increase in analyte level at a rate that will result in violation of an actual high analyte threshold (this alarm can also require a recent measurement to be greater than a predicted high analyte threshold that indicates proximity to the actual high analyte threshold). Other examples exist as well.

In some embodiments, the alarm indication transmitted from OBD 102 over RF link 142 can include data representative of the one or more analyte measurements that served to generate the alarm. Upon receipt of the alarm indication from OBD 102, reader 120 can process the analyte data if necessary and notify the user of the alarm condition (e.g., by display of the alarm indication (e.g., low analyte level, high analyte level, etc.) on display 122, by generation of an audible notification, and/or by generation of a tactile notification (e.g., vibration), and display the received analyte data. The user can then take appropriate corrective action.

In other embodiments, the alarm indication transmitted from OBD 102 over RF link 142 can indicate the presence of an alarm, but not include data representative of the one or more analyte measurements that served to generate the alarm. Upon receipt of the alarm indication from OBD 102, reader 120 can notify the user (e.g., by display of an alarm indication on display 122, generation of an audible notification, and/or generation of a tactile notification (e.g., vibration)) of the existence of an alarm condition. In some embodiments, the type of alarm condition (e.g., low analyte level, high analyte level, etc.) is transmitted without the underlying analyte data. In other embodiments, only an indication of the alarm condition is transmitted but without specifying the alarm type (e.g., a generic alarm indication generic to all alarms). In these embodiments, reader 120 can display a recommendation to the user to scan OBD 102 to obtain additional information, such as the user's most recent analyte measurement and/or the one or more analyte measurements that caused generation of the alarm. Other information can also be obtained such as additional measurements going back further in time (historical measurements), trend information, and information regarding any potential analyte event (e.g., low glucose, high glucose, rapid rise in glucose, rapid fall in glucose, etc.). The user can then take appropriate corrective action. In still other embodiments, the transmitted alarm indication can specify whether the alarm is related to an analyte measurement or some other factor (e.g., failure of the sensor, expiration of the sensor, etc.), and reader device 120 can generate an alarm for the user that informs the user of the alarm type.

### Example Embodiments of Establishing, Maintaining, and Reestablishing Synchronization

RF link 142 is preferably a synchronized link where communications between OBD 102 and reader 120 are sent and received at periodic time intervals or points in time known to each device. Provided herein are a number of example embodiments for establishing, maintaining, and/or reestablishing synchronization between OBD 102 and reader 120 communicating across RF link 142. In many embodiments, RF link 142 operates in the UHF frequency band. Although not limited to such, these example embodiments will be described with reference to an example RF link 142 operating in the UHF band.

OBD 102 can transmit data or information (e.g., such as the alarm indications and/or analyte data described above) in a burst over a short period of time relative to the communication period. Having the receiver always listening for data packets on a RF link (UHF for example) leads to high battery consumption and is not desirable. In many embodiments the receiver monitors the UHF band for receipt of a transmission during a limited time period. There can be challenges in synchronizing data transmission between devices, such as from OBD 102 to reader 120. There can be variations between different OBDs 102, such as component variations, that can lead to differences in determining the exact time at which data is transmitted. There can be environmental variations, such as temperature, that can also lead to differences in determining transmission times. If synchronization is lost, then the system requires a technique or mechanism to reestablish communication and synchronization. Example embodiments described here can be used to address these issues that could impact the synchronization of data communication.

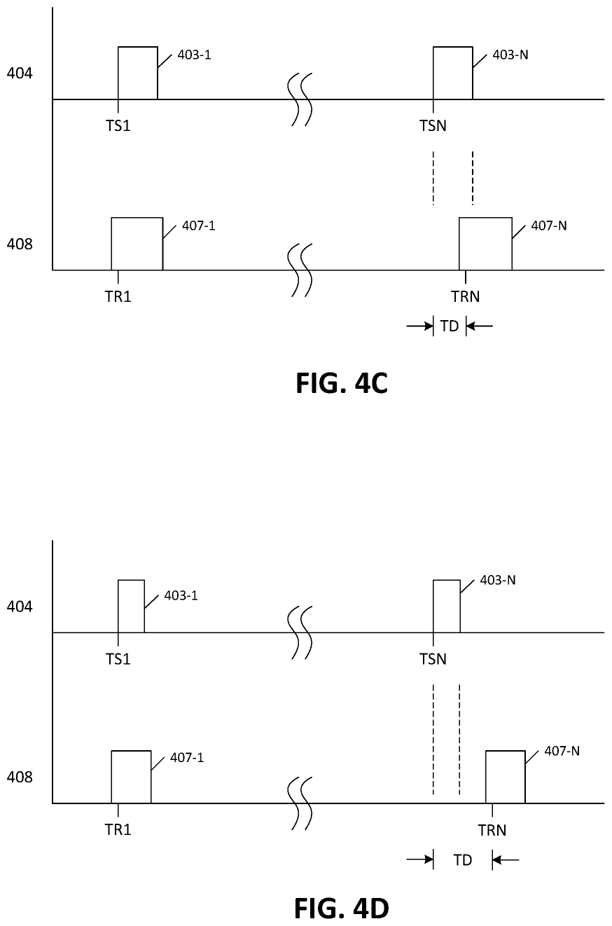

FIG. 4A is a timing diagram depicting an example of synchronous communication within system 100. Waveform 402 represents a clock or other timing signal generated by timing circuitry 310 within OBD 102. OBD 102 can use clock 402 to determine the appropriate times at which OBD 102 should transmit over RF link 142. Waveform 404 depicts transmissions 403 that occur from OBD 102. Each of these transmissions 403 occurs at a time (Ts) determined by OBD 102 to be the appropriate transmission time for communication over link 142. Here, four sequential transmissions 403-1, 403-2, 403-3, and 403-4 occur at times TS1, TS2, TS3, and TS4, respectively.

In some embodiments, a transmission 403 occurs at each sequential transmission time (Ts) (e.g., as shown in FIG. 4A). The period of time between sequential transmissions times is referred to herein as the communication interval (CI) or communication period. Each transmission 403 can include one or more data bits (e.g., flags) that indicate whether the transmission 403 is an actual alarm indication or whether the transmission 403 is merely a maintenance transmission used to maintain synchronization, or otherwise. The length (TP) of each transmission 403 can be fixed, such that the devices in system 100 are programmed with the determined length of each transmission 403. In other embodiments, such as that depicted in FIG. 4A, the length of TP can be variable and dependent on the quantity of data to be transmitted (e.g., compare TP1 to TP2).

Likewise, the CI has a target length that is constant, but that length can be selected as desired for the particular implementation. CI can be one or more orders of magnitude longer than the TP. For example, the TP can last only a fraction of a second, such that all data is transmitted in that fraction of a second, and the CI can be 30 seconds, one minute, two minutes, three minutes, five minutes, ten minutes and so forth. The CI can be selected such that a transmission 403 can occur each time a sensor measurement is made or recorded. For example, if OBD 102 is programmed to collect an analyte measurement every one minute then CI can also be one minute. In other embodiments, CI can be selected to be longer than the interval between successive sensor measurements. For example, if OBD 102 collects an analyte measurement every two minutes then CI may be, e.g., ten minutes.

Waveform 406 represents a clock or other timing signal generated by timing circuitry 219 within reader 120. Reader 120 can use clock 406 to determine the appropriate times at which reader 120 should power its second communication circuitry 242 to monitor for receipt of a transmission 403 over RF link 142. Clocks 402 and 406 can be the same frequency or different frequencies (as shown here). Waveform 408 includes multiple receiving periods or receiving windows 407, which are periods of time during which reader 120 actively monitors for receipt of a transmission. The length of a receiving window 407 is denoted as RP. If a transmission 403 is sent outside of a receiving window 407, then that transmission will not be received by reader 120. Reader 120 can determine the times at which it expects to receive a transmission 403 from OBD 102. These times are indicated as TR1, TR2, TR3, and TR4 (corresponding to TS1, TS2, TS3, and TS4 respectively). Reader 120 can use these expected transmission times TR to determine when each receiving window 407 should occur. As shown here, receiving windows 407-1, 407-2, 407-3, and 407-4 are enabled at times including each expected transmission time TR1, TR2, TR3, and TR4, respectively. Also as shown here, each receiving window 407 can begin just prior to TR in order to allow for a margin of error if reader 120 determines TR to occur at a slightly later time than the corresponding TS determined by OBD 102.

An initial synchronization event 401 can occur for use as a reference point by both devices 102 and 120 to determine a starting time TO from which to begin the CI, by OBD device 102 to determine the transmission times (TS), and by reader 120 to determine the expected transmission times (TR). Synchronization event 401 can be in the form of a handshake with a communication from reader 120 to OBD 102 and a return acknowledgment transmission from OBD 102 back to reader 120 (or vice-versa). A handshake type exchange is indicated by the hashed region 401 in FIG. 4A. In other embodiments, synchronization event 401 can be a single transmission either from OBD 102 to reader 120 or from reader 120 to OBD 102. The synchronization event 401 can be performed over either link 141 (such as during an initial activation scan of OBD 102) or over link 142, e.g., after activation of OBD 102, as part of a predetermined link synchronization routine.

In the embodiment described with respect to FIG. 4A, a transmission 403 occurs at each TS. Each transmission 403 can include a flag (e.g., one or more data bits) that indicates whether or not an alarm condition exists. Those transmissions 403 that do not indicate the existence of an alarm condition, e.g., a maintenance transmission, can be used as verification that synchronization across link 142 has been maintained.

Transmissions 403 can also occur on an ad hoc basis, or only as needed, or with a combination of ad hoc transmissions and regular period transmissions. For example, in some embodiments a transmission 403 occurs only if OBD 102 determines that a transmission is warranted, e.g., if an alarm condition has been detected. This may lead to long periods of time without the exchange of transmissions 403 so, in these embodiments, a transmission 403 can be periodically generated even if no alarm condition exists. This maintenance transmission can be used to verify that OBD 102 and reader 120 are still in synchronous communication. The period of time between maintenance transmissions can be referred to as the maintenance period, which can be every X (e.g., two, three, four, five, ten, etc.) communication periods. For example, if no transmission has been made after X−1 transmission times (Ts), then at the Xth transmission time OBD 102 can send a transmission that is flagged as either an alarm condition (if one has been triggered) or a maintenance transmission. Reader 120 can be programmed to anticipate receipt of a transmission after X−1 silent transmission times have past and, at the Xth transmission time, can use receipt of the transmission as verification of the synchronous link. Reading the data bits in the transmission will indicate whether the transmission is a maintenance transmission or an actual alarm indication (at which point an alarm can be generated). If no transmission is received at the Xth transmission time then reader 120 can determine that the synchronous link has been lost and generate a notification to the user and/or enter a procedure to reestablish synchronization.

FIG. 4B is a timing diagram depicting an example embodiment where X=10. A transmission 403-1 from OBD 102 occurs at TS1 and is received by reader device 120 during receiving window 407-1. Nine (i.e., X−1) consecutive transmission times then pass without another transmission from OBD 102. If no alarm condition occurs, a transmission 403-2 is forced to occur at TS11, which is received by reader 120 during receiving window 407-11. Transmission 403-2 can include a flag indicating that it is a maintenance transmission.

In some embodiments, the length of RP is constant (or fixed) and reader 120 will continue to listen for transmission during the entire fixed RP. In other embodiments, the length (RP) of each receiving window 407 can be variable. For instance, as depicted in FIG. 4B, the length of receiving period (e.g., RP1) can continue as long as reader 120 is receiving data, with the receiving period ending after a predetermined time in which no data is received (or if a maximum allotted amount of time, as programmed in the system, has been reached). If after a certain amount of time no data has been received, then receiving period 407 can be terminated, resulting in a shorter receiving period (e.g., RP3) and conserving power. In order to conserve battery power, it is desirable that reader device 120 listen for transmissions during the shortest time feasible. For example, if the CI is 60 seconds, then it is beneficial for reader device 120 to only listen for less than one second, less than two seconds, less than five seconds, or the like during each 60 second interval, even if RP is fixed.

In the foregoing embodiments, whether transmissions occur every CI or at a lesser rate, due to structural and/or environmental factors, variations in device performance can result in small differences between the time (TS) at which OBD 102 sends a transmission and the corresponding time at which reader 120 determines that the transmission should be received (TR). In some embodiments, variations in the fabrication of the components and/or circuitry of timing circuitry 310 can cause different OBDs 102 to determine different times of transmission (TS). For example, variations in performance of a linear oscillator such as an RC oscillator can cause timing circuitry 310 of one OBD 102 to operate faster than that of another OBD 102, even though the timing circuitries 310 of both OBDs 102 are intended to operate at the same frequency. Also, the temperature at which a particular OBD 102 is operating can also affect the speed of the clock, and this temperature variable may not similarly apply to the receiving reader 120. Also, a high degree of clock variation can affect the timing of the data in its un-modulated or demodulated form, making it more difficult or impossible to decode and read the received data at reader device 120.

FIGS. 4C-4D are timing diagrams depicting examples where OBD 102 and reader 120 are generally synchronized at TS1 and TR1, but after a large number (N) of CIs, the transmission time TSN determined by OBD 102 is different than the transmission time (TRN) determined by reader 120. The difference between TSN and TRN is denoted as TD. In FIG. 4C, the OBD transmission at TSN still overlaps the receiving period (RPN) and part of the data is cut off, whereas in FIG. 4D the determined transmission times are far enough apart that no overlap is present and no data is received.

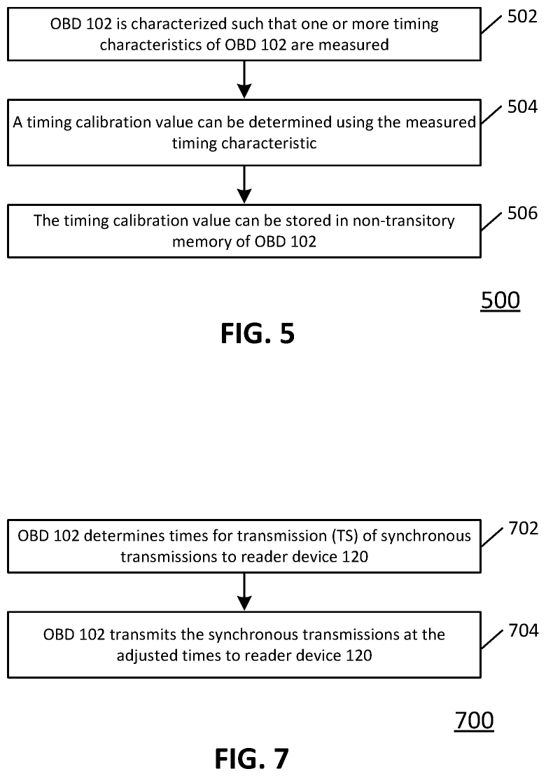

In some embodiments, a timing calibration value that is specific to a particular OBD 102 can be determined and used to adjust the timing of communications over RF link 142 to maintain synchronization. FIG. 5 is a flow diagram depicting an example embodiment of determining a timing calibration value. At 502, OBD 102 is characterized such that one or more timing characteristics of OBD 102 are measured. This measurement can be performed by the manufacturer during or after assembly of OBD 102. The timing characteristic can be a characteristic of OBD 102 that determines the actual rate of a timing signal produced by timing circuitry 310. In some embodiments, the timing characteristic is the actual frequency of an oscillator of timing circuitry 310 and/or the actual frequency of timing circuitry 310 itself.

At 504, a timing calibration value can be determined using the measured timing characteristic. This timing calibration value can take various forms. For example, the timing calibration value can be the measured frequency of the oscillator of timing circuitry 310 or the measured frequency of timing circuitry 310. The timing calibration value can be an offset (positive or negative) of the measured frequency from a predetermined target, e.g., ideal or desired, frequency of the measured component. The timing calibration value can also be expressed as a time. For example, the timing calibration value can be a measured length of the communication interval (CI) as executed by OBD 102 during sample operation. The timing calibration value can instead be an offset (positive or negative) of the measured CI from a predetermined target CI. The timing calibration value can be in the form of a code that corresponds to a particular value. The code can be translated to the corresponding value by use of a lookup table (e.g., stored in memory of reader 120).

Once the timing calibration value has been determined, in many embodiments the timing calibration value can optionally be stored in non-transitory memory 308 (e.g., non-volatile RAM or flash) of OBD 102 at 506. The timing calibration value can be stored by writing the calibration value directly to memory 308 if the leads of the chip having memory 308 are readily accessible. In other embodiments, if the electronics of OBD 102 are assembled (e.g., on a main PCB), then the timing calibration value can be written to memory 308 using a serial access port, such as a boundary scan or JTAG interface, or can be written to memory 308 using a wireless transmission that can be received using communication circuitry 341 or 342, and then processor 306 can cause the timing calibration value to be written to memory 308. OBD 102 can complete the manufacturing process (if necessary) and be provided to end-users. In some embodiments, instead of storing the calibration value in memory 308, the calibration value can instead be printed on the packaging of OBD 102 or directly on the housing of OBD 102. In these embodiments, the user can read the calibration value and input the value into reader 120, or alternatively use reader 120 to automatically obtain the timing calibration value (e.g., by optically scanning a barcode on the packaging or housing for OBD 102). In other embodiments, the timing calibration value can be uploaded to trusted computer system 180 and then subsequently retrieved by reader device 120. In these embodiments, reader device 120 can obtain an identifier for OBD 102 (either by scanning OBD 102 or by manually or automatically obtaining the identifier, etc.) and transmit it to trusted computer system 180 which can then locate the corresponding timing calibration value and communicate it back to reader device 120 over a network 190.

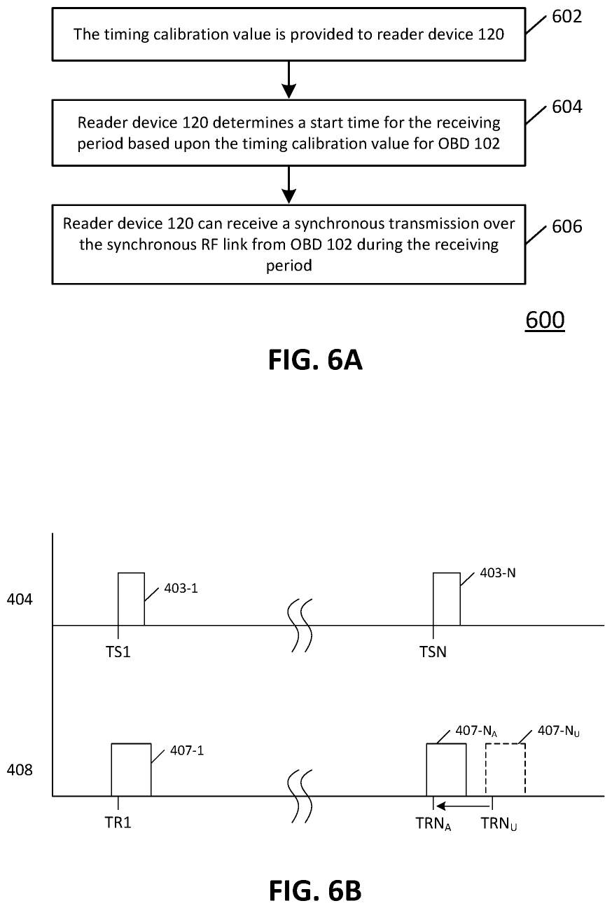

The timing calibration value can be used to adjust the times at which transmissions are sent or the times at which transmissions are expected to be received within system 100. FIG. 6A is a flow diagram depicting an example embodiment of method 600 where reader device 120 uses the timing calibration value to determine the appropriate times at which to enable the receiving window.

At 602, the timing calibration value is provided to reader device 120. In some embodiments, the timing calibration value is transmitted from OBD 102 to reader device 120 when reader device 120 performs an initial activation scan of OBD 102 over near field link 141. In other embodiments, the timing calibration value can be downloaded to reader 120 from trusted computer system 180 or otherwise manually or automatically input to reader 120 as described above. Reader device 120 may have already been used with other OBDs 102 having different timing calibration values.

OBD 102 and reader device 120 can each be programmed to recognize a synchronization event for use in establishing TO. In embodiments where the timing calibration value is transmitted from OBD 102 to reader device 120 over near field link 141, the sending of this transmission and receipt thereof can be used as the synchronization event. Other transmissions over near field link 141 can also be used as the synchronization event. In other embodiments, after activation of OBD 102, reader device 120 can enable a continuous receiving window 407 and monitor for receipt of a first transmission 403 from OBD 102 over RF link 142, the receipt of which can be used as the synchronization event.

At 604, reader device 120 determines a start time for receiving period 407 based upon the timing calibration value for OBD 102, e.g., by execution of software instructions by processor 206. If the timing calibration value is in the form of a code, then reader processor 206 can translate the code into a usable value by reference to a lookup table or other data structure stored in reader 120. If the timing calibration value is in the form of a measured frequency (or frequency offset), processor 206 can determine the first expected transmission time TR1 using the measured frequency (or frequency offset) and the target CI. For example, if the target CI is 60 seconds, the target frequency of OBD 102 is 10 Mhz, and the measured frequency of OBD 102 is 9.95 Mhz (corresponding to an offset of −0.05 Mhz), then the expected transmission time TR of the first transmission 403 will be 60.302 seconds after TO. If the timing calibration value is in the form of a measured CI (e.g., 59.995 seconds) or offset to a target CI (e.g., −0.005 seconds), then processor 206 can calculate TR directly using the measured CI.

At 606, reader device 120 can receive transmission 403 over RF link 142 from OBD 102 during receiving period 407. At the expiration of receiving period 407 reader device 120 does not use power to monitor for a transmission from OBD 102. Using the calculated first transmission time TR as a new value for TO, reader 120 can then calculate the next transmission time TR2 and proceed to enable receiving period 407 for a span encompassing that transmission time TR2. This process can repeat itself until OBD 102 reaches the end of its lifespan. FIG. 6B is a timing diagram depicting an example similar to that of FIG. 4B, except the position of the Nth receiving window 407-N has been adjusted using method 600. The unadjusted Nth receiving window 407-Nu occurring around the unadjusted expected transmission time TRNu is adjusted to a new position 407-NA by application of method 600. The new adjusted position occurs around an adjusted expected transmission time TRNA that is synchronized with TSN.

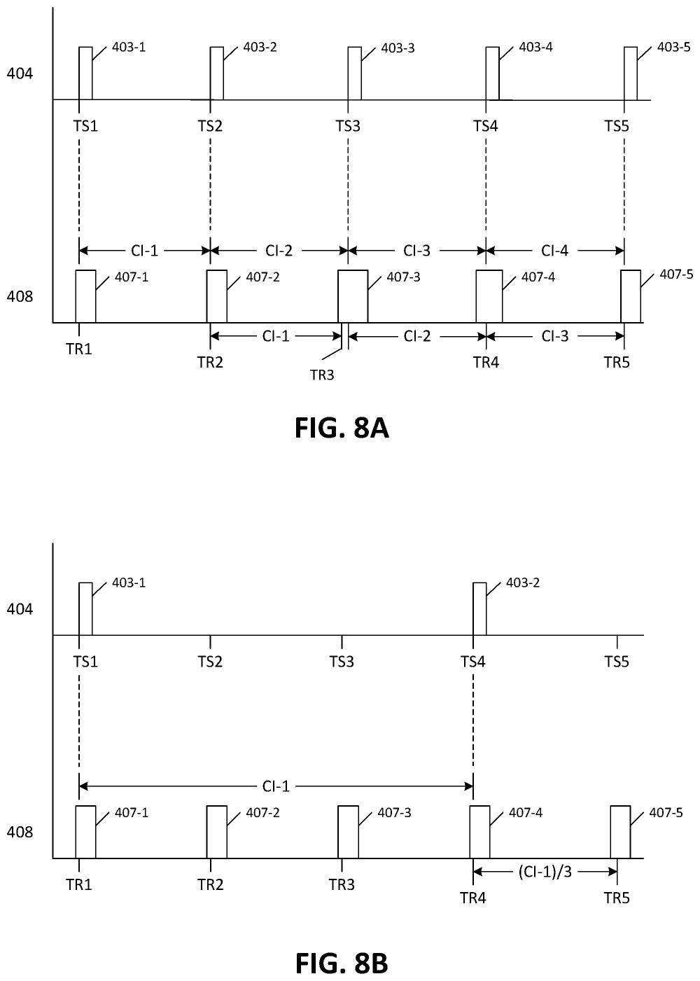

FIG. 7 is a flow diagram depicting an example embodiment of a method 700 where OBD 102 uses the timing calibration value to determine the appropriate times at which to send a transmission 403. At 702, OBD 102 determines times for transmission (TS) of synchronous transmissions 403 to the reader device. Processor 306, executing software instructions, can determine the times for transmission using a timing calibration value stored in memory 308. In this embodiment, the timing calibration value is used to adjust the transmission times so that they occur at or close to the ideal target times. For example, the frequency of OBD 102 can be measured as described above with respect to FIG. 5. The offset of this measured frequency from the ideal target frequency can be determined and translated to an offset in time that can be stored as the timing calibration value within memory 308. Alternatively, the offset in time can be measured by comparing an actual time of transmission by OBD 102 to the ideal target transmission time (or the actual CI to the target CI). This offset in time can be used by OBD 102 to adjust each determined time of transmission TS. For example, if the offset in time is +0.01 seconds, then OBD 102 can delay each synchronous transmission 403 by 0.01 seconds. At 704, OBD 102 transmits the synchronous transmissions 403 at the adjusted times to reader device 120. Thus, in this embodiment, reader device 120 can operate with multiple successive OBDs 102 without adjusting its own timing.

OBD 102 and reader device 120 can each be programmed to recognize a synchronization event for use in establishing TO. In some embodiments, a transmission (such as an activation command) received by OBD 102 from reader 120 over near field link 141 is used as the synchronization event. In other embodiments, after activation of OBD 102, reader device 120 can enable a continuous receiving window 407 and monitor for receipt of a first transmission 403 from OBD 102 over RF link 142, the transmission and receipt of which can be used by the respective devices 102 and 120 as the synchronization event.

As mentioned, environmental factors such as temperature can cause timing circuitry 310 to run faster or slower than a target rate. To account for this, OBD 102 can include a temperature sensor 311 (FIG. 3) that can monitor temperature continuously (e.g., at closely spaced discrete intervals). This temperature information can then be used to adjust the times of transmission TS or expected times of receipt TR. In the embodiments of method 600 and 700 described above, the timing calibration value can also include this temperature information. The device that utilizes the timing calibration value to adjust timing (e.g., reader 120 in method 600 and OBD 102 in method 700), can also adjust timing based on this temperature information. In some embodiments, the timing calibration value can include only this temperature information and the corresponding timing adjustments are made on temperature information alone.

The temperature information can be in the form of multiple discrete temperature measurements, a central tendency (e.g., mean or median) of the multiple discrete temperature measurements, an integral of temperature over time, or the like. Each of the foregoing can be expressed as an offset from a target temperature as well. The processing circuitry can increase or decrease the determined times (TS or TR) based on the temperature information and an algorithmic correlation of temperature to speed of timing circuitry 310. For example, some oscillators perform at a target frequency when ambient temperature is close to room temperature (or other target temperature), but the actual frequency will decrease as the ambient temperature moves away from room temperature (colder or hotter). In such embodiments, OBD 102 will tend to transmit later than expected as temperature decreases or increases away from room temperature. Other embodiments of timing circuitry 310 can exhibit different behaviors. A predetermined temperature compensating algorithm can be stored in memory and executed by the respective processor for this purpose.

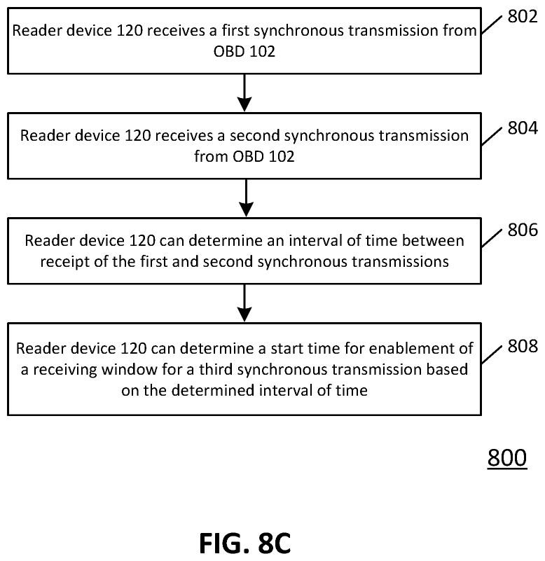

In some embodiments, reader device 120 can monitor the actual CI between transmissions 403 from OBD 102, and adjust the estimated time of receipt TR based on the monitored CI length. FIG. 8A is a timing diagram depicting an example embodiment where reader device 120 monitors the actual CI between transmissions 403. Reader device 120 can determine a length of time CI−1 from receipt of transmission 403-1, e.g., a time at which a first packet of transmission 403-1 is received, to receipt of a subsequent transmission 403-2. If the length of time CI−1 is within an expected range indicating that it corresponds to one communication interval, then reader device 120 can use the length of time CI−1 to determine the expected time of receipt TR3 for the next transmission 403-3 and set the start time of receiving window 407-3 accordingly (e.g., the time of receipt of transmission 403-2 plus CI−1). Likewise, reader device 120 can determine a length of time CI−2 between receipt of transmission 403-2 and receipt of transmission 403-3 and use CI−2 to determine the expected time of receipt TR4 for the next transmission 403-4, and so forth. In this manner, reader device 120 can adjust occurrence of receiving windows 407 based on the most measured characteristics of the transmissions from OBD 102.

In this embodiment, the length of the CI used to set the next expected time of receipt TR is the most recently measured CI. In other embodiments, the length of the CI used to set the next expected time of receipt TR can be an average of a predetermined number of preceding CI measurements (e.g., two, three, four, or more).

FIG. 8B is a timing diagram depicting another example embodiment where reader device 120 monitors the actual CI between transmissions 403. Here, OBD 102 does not send a transmission 403 at each transmission time TS. Reader device 120 can determine a length of time CI−1 from receipt of transmission 403-1 to receipt of a subsequent transmission 403-2. The length of time CI−1 can be divided by a target CI (e.g., TS2−TS1) to determine the number of communication intervals that have transpired, which in the embodiment of FIG. 8B is three. The length of time CI−1 can be divided by the number of CIs in this value can be used to set the start time of receiving window 407-5 (e.g., the time of receipt of transmission 403-2 plus (CI−1)/3). The same value can be used to set subsequent TRs until another transmission 403 is received at which point the process can be repeated.

FIG. 8C is a flow diagram depicting an example embodiment of a method 800 for continually monitoring the actual CI between transmissions. At 802, reader device 120 receives a first synchronous transmission (403-1) from OBD 102. At 804, reader device 120 receives a second synchronous transmission (403-2) from OBD 102. At 806, reader device can determine an interval of time (CI−1) between receipt of the first and second synchronous transmissions. At 808, reader device 120 can determine a start time for enablement of a receiving window (407) for a third synchronous transmission based on the determined interval of time. If the first and second synchronous transmissions occurred at one communication interval, then the start time for enablement of the receiving window can be the time of receipt of the second synchronous transmission plus the determined interval of time (like the embodiment of FIG. 8A). If the first and second synchronous transmissions occurred over a longer period of time encompassing multiple communication intervals then an estimated value for a single communication interval can be determined and used to set the start time of the next receiving window (like the embodiment of FIG. 8B). In the embodiments described with respect to FIGS. 8A-8C, the start time of the first receiving window 407-1 can be determined using a synchronization event 401 (as described elsewhere herein plus the length of a target CI).

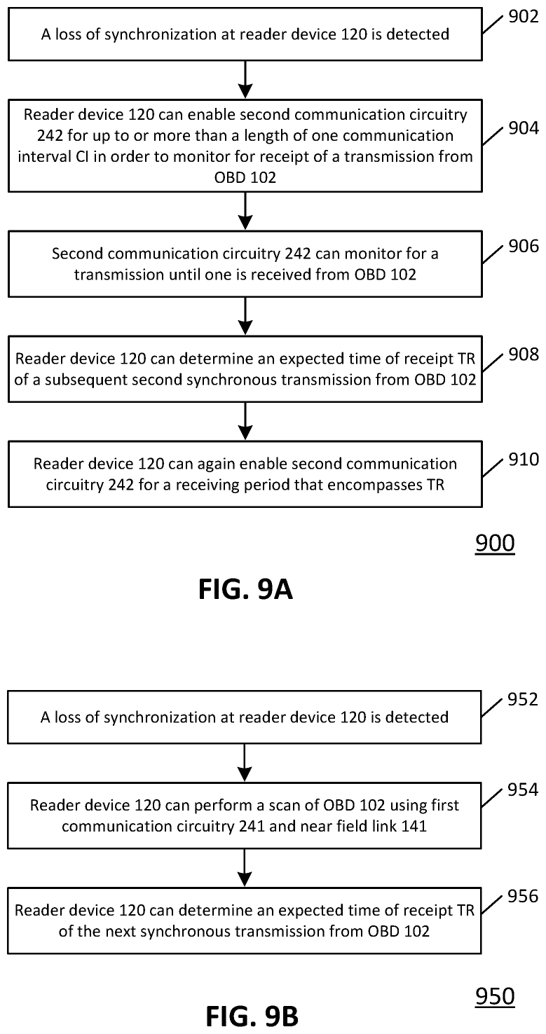

In certain scenarios system 100 may lose synchronization between OBD 102 and reader 120. Example embodiments are provided herein for establishing or reestablishing synchronization after its loss. FIG. 9A is a flow diagram depicting an example embodiment of a method 900 for establishing synchronization. At 902, a loss of synchronization at reader device 120 is detected. The loss of synchronization can be signified by failure to receive a transmission 403 during one or more successive receiving windows 407. Once reader device 120 has determined that the loss of synchronization has occurred, then at 904 reader device 120 can enable second communication circuitry 242 for up to or more than a length of one communication interval CI in order to monitor for receipt of a transmission from OBD 102. Second communication circuitry 242 can monitor for a transmission 403 until one is received at 906. Upon receipt, second communication circuitry 242 can be disabled (e.g., power can be disapplied). In some embodiments, the resynchronization procedure can include lengthening the duration of receiving window 407 iteratively. For example, receiving window 407 can be lengthened by a predetermined number of seconds or percentage in a first CI and then, if no transmission is received, the receiving window can be lengthened again in similar fashion at the next CI. This process can proceed iteratively until a transmission is received or until reader device 120 has expanded its listening window to the full CI.

At 908, reader device 120 can determine an expected time of receipt TR of a subsequent second synchronous transmission from OBD 102. At 910, reader device 120 can again enable second communication circuitry 242 for a receiving period 407 that encompasses TR. This receiving period 407 is preferably less than a communication interval CI. If OBD 102 is sending synchronous transmissions then the next transmission should be received during this receiving period 407. OBD 102 and reader device 120 can then continue with synchronous communication according to any of the embodiments described herein.

FIG. 9B is a flow diagram depicting another example embodiment of a method 950 for establishing synchronization. At 952, reader device 120 detects a loss of synchronization. The loss of synchronization can be signified by failure to receive a transmission 403 during one or more successive receiving windows 407. Once reader device 120 has determined that the loss of synchronization has occurred, then at 954 reader device 120 can perform a scan of OBD 102 using first communication circuitry 241 and near field link 141. In some embodiments, once reader device 120 has determine that the loss of synchronization has occurred, reader device 120 can output a notification or instruction (e.g., such as audibly, visually, and/or tactilely) for a user to perform the scan. Performance of the scan by the user can include bringing reader device 120 into close proximity (e.g., within a distance of twelve inches) from OBD 102, transmitting a communication from reader device 120 to OBD 102 across near field link 141, and receiving a response communication at reader device 120 from OBD 102. The communication transmitted from reader device 120 can indicate to OBD 102 that a loss of synchronization has occurred at which point OBD 102 can implement a routine to reestablish synchronization, for example by re-setting the timing of its synchronous transmissions 403 based on a new synchronization event. In some embodiments, the new synchronization event for OBD 102 can be the transmission of the response communication back to reader device 120 as part of the scan. The new synchronization event for reader 120 can be receipt of the response communication during the scan.

At 956, reader device 120 can determine an expected time of receipt TR of the next synchronous transmission from OBD 102. This determination can be made according to any of the embodiments described herein, e.g., such as by use of a timing calibration value of OBD 102. Reader device 120 can enable a receiving window 407 at the next expected time of receipt TR, at which point a synchronous communication 403 should be received and synchronization reestablished.

The embodiments described herein are restated and expanded upon in the following paragraphs without explicit reference to the figures. In many embodiments, a method of communication in an analyte monitoring system including an on body device and a reader device is provided, the method including: receiving, at the reader device, a timing calibration value from the on body device; determining, by the reader device, a start time for a receiving period based upon the timing calibration value of the on body device; and receiving a transmission from the on body device during the receiving period.

In some embodiments, the on body device and the reader device are adapted to communicate synchronously at communication intervals, where the length of each communication interval as determined within the system is constant. The method can further include determining, by the reader device, a start time for a plurality of receiving periods based upon the timing calibration value from the on body device, where each receiving period occurs after a communication interval. The receiving period can be a length of time less than a communication interval where the reader device uses power to monitor for the transmission from the on body device. In some embodiments, the reader device does not use power to monitor for the transmission from the on body device at times outside of the receiving period.

In some embodiments, the timing calibration value includes a clock frequency, a clock frequency offset, or a length of time. In some embodiments, the timing calibration value includes a length of a communication interval. In some embodiments, the timing calibration value includes an offset to a target timing value. In some embodiments, the timing calibration value includes temperature information.

In many embodiments, the on body device includes an analyte sensor that can be positioned in the body of a user.

In many embodiments, the reader device includes first communication circuitry configured to communicate with inductive coupling, and second communication circuitry. The timing calibration value can be received by the reader device using the first communication circuitry, and the transmission from the on body device can be received using the second communication circuitry. In many embodiments, the second communication circuitry is configured to communicate within an ultra-high frequency (UHF) band and the transmission from the on body device is received within an ultra-high frequency (UHF) band. In many embodiments, the first communication circuitry is configured to communicate according to a Near Field Communication (NFC) protocol.

In many embodiments, the method further includes performing a synchronization event that synchronizes the on body device and the reader device. In many embodiments, the reader device uses receipt of the timing calibration value from the on body device as a synchronization event.

In many embodiments, a reader device for use in an analyte monitoring system is provided, the reader device including: wireless communication circuitry; processing circuitry communicatively coupled with the wireless communication circuitry; and a non-transitory memory on which is stored a plurality of instructions that, when executed by the processing circuitry, cause the processing circuitry to: read a timing calibration value received from an on body device; determine a start time for a receiving period based upon the timing calibration value of the on body device; and enable the wireless communication circuitry to receive a transmission from the on body device during the receiving period.

In many embodiments, the plurality of instructions, when executed by the processing circuitry, cause the processing circuitry to cause communication with the on body device synchronously at communication intervals, where the length of each communication interval is constant. The plurality of instructions, when executed by the processing circuitry, can also cause the processing circuitry to determine a start time for a plurality of receiving periods based upon the timing calibration value from the on body device, where each receiving period occurs after a communication interval. The plurality of instructions, when executed by the processing circuitry, can also cause the processing circuitry to apply power to the wireless communication circuitry during the receiving period. The plurality of instructions, when executed by the processing circuitry, can cause the processing circuitry to not apply power to a receiver of the wireless communication circuitry at times outside of the receiving period.

In many embodiments, a method of communication in an analyte monitoring system including an on body device and a reader device is provided, the method including: transmitting, by the on body device, a timing calibration value to the reader device; and transmitting synchronous transmissions to the reader device.

In some embodiments, the synchronous transmissions are transmitted at communication intervals, the length of each communication interval as determined with the system being constant.

In some embodiments, the timing calibration value is derived from a characterization of the on body device. In some embodiments, the timing calibration value includes a clock frequency of the on body device, a clock frequency offset of the on body device, or a length of time. In some embodiments, the timing calibration value includes an offset to a target timing value, the offset having been determined by a characterization of the on body device. In some embodiments, the on body device includes a temperature sensor, and the method further includes measuring temperature with the temperature sensor, and the timing calibration value includes measured temperature information.

In some embodiments, the method further includes performing a synchronization event that synchronizes the on body device and the reader device. In some embodiments, the on body device uses transmission of the timing calibration value to the reader device as a synchronization event.

In many embodiments, an on body device for use in an analyte monitoring system is provided, the on body device including: wireless communication circuitry; processing circuitry communicatively coupled with the wireless communication circuitry; and a non-transitory memory on which is stored a plurality of instructions that, when executed by the processing circuitry, cause the processing circuitry to: cause transmission of a timing calibration value to a reader device; and cause synchronous transmissions to the reader device.

In many embodiments, a method of calibrating synchronous timing within an analyte monitoring system is provided, the method including: measuring a timing characteristic of an on body device; determining a timing calibration value based on the measured timing characteristic; and storing the timing calibration value in non-transitory memory of the on body device.

In some embodiments, the method further includes providing the on body device to a user after storing the timing calibration value in the non-transitory memory of the on body device. The timing calibration value can be written to the non-transitory memory of the on body device using an RF transmission.

In some embodiments, the timing calibration value is derived from a characterization of the on body device. In some embodiments, the timing calibration value is a clock frequency of the on body device, a clock frequency offset of the on body device, or a length of time. In some embodiments, the timing calibration value is an offset to a target timing value, the offset having been determined from the measured timing characteristic.

In some embodiments, the on body device is programmed to transmit the timing calibration value to a reader device.

In some embodiments, the on body device is programmed to adjust the timing of synchronized transmissions to a reader device using the timing calibration value.

In many embodiments, a method of communication in an analyte monitoring system including an on body device and a reader device is provided, the method including: determining, by the on body device, times for transmission of synchronous transmissions to the reader device, where the times for transmission are determined using timing circuitry of the on body device and a timing calibration value; and transmitting the synchronous transmissions to the reader device.

In many embodiments, an on body device for use in an analyte monitoring system is provided, the on body device including: timing circuitry; wireless communication circuitry; processing circuitry communicatively coupled with the wireless communication circuitry; and a non-transitory memory on which is stored a plurality of instructions that, when executed by the processing circuitry, cause the processing circuitry to: determine times for transmission of synchronous transmissions to a reader device, where the times for transmission are determined using the timing circuitry and a timing calibration value; and cause transmission of the synchronous transmissions to the reader device.

In some embodiments, the plurality of instructions, when executed by the processing circuitry, cause the processing circuitry to cause transmission of the each synchronous transmission at the end of a communication interval, the length of the communication interval being constant.

In many embodiments, a method of communication in an analyte monitoring system including an on body device and a reader device is provided, the method including: receiving, at the reader device, a first synchronous transmission from the on body device; receiving, at the reader device, a subsequent second synchronous transmission from the on body device; determining, by the reader device, an interval of time between receipt of the first and second synchronous transmissions; and determining, by the reader device, a start time for enablement of a receiving window for a third synchronous transmission based on the determined interval of time, where the receiving window is a limited period of time less than the determined interval of time.

In some embodiments, the method further includes: enabling the receiving window at the start time; receiving the third synchronous transmission from the on body device; and disabling the receiving window. In some embodiments, the interval of time is a first interval of time and the receiving window is a first receiving window, and the method further includes: determining, by the reader device, a second interval of time between receipt of the second and third synchronous transmissions; and determining, by the reader device, a start time for enablement of a second receiving window for a fourth synchronous transmission based on the determined second interval of time, where the second receiving window is a limited period of time less than the determined second interval of time.

In some embodiments, the start time for enablement of the receiving window is a time of receipt of the second synchronous transmission plus the determined interval of time. In some embodiments, the start time for enablement of the receiving window is a time of receipt of the second synchronous transmission plus an estimated communication interval length of time, where the estimated communication interval length of time is the determined interval of time divided by an estimated number of communication intervals.

In many embodiments, a reader device for use in an analyte monitoring system is provided, the reader device including: wireless communication circuitry; processing circuitry communicatively coupled with the wireless communication circuitry; and a non-transitory memory on which is stored a plurality of instructions that, when executed by the processing circuitry, cause the processing circuitry to: determine an interval of time between receipt of a first synchronous transmission from an on body device and receipt of a second synchronous transmission from the on body device; and determine a start time for enablement of a receiving window for a third synchronous transmission based on the determined interval of time, where the receiving window is a limited period of time less than the determined interval of time.

In some embodiments, the plurality of instructions, when executed by the processing circuitry, cause the processing circuitry to: enable the wireless communication circuitry at the start time; and disable the wireless communication circuitry after receipt of the third synchronous transmission.

In some embodiments, the interval of time is a first interval of time and the receiving window is a first receiving window, and where the plurality of instructions, when executed by the processing circuitry, cause the processing circuitry to: determine a second interval of time between receipt of the second and third synchronous transmissions; and determine a start time for enablement of a second receiving window for a fourth synchronous transmission based on the determined second interval of time, where the second receiving window is a limited period of time less than the determined second interval of time.

In some embodiments, the start time for enablement of the receiving window is a time of receipt of the second synchronous transmission plus the determined interval of time. In some embodiments, the start time for enablement of the receiving window is a time of receipt of the second synchronous transmission plus an estimated communication interval length of time, where the estimated communication interval length of time is the determined interval of time divided by an estimated number of communication intervals.

In many embodiments, a method of reestablishing synchronization in an analyte monitoring system including an on body device and a reader device is provided, where synchronous transmissions occur at a communication interval, the method including: detecting a loss of synchronization at the reader device; enabling communication circuitry of the reader device for a length of the communication interval to monitor for receipt of a first synchronous transmission from the on body device; receiving, at the reader device, a first synchronous transmission from the on body device; disabling the communication circuitry of the reader device; determining, by the reader device, an expected time of receipt of a subsequent second synchronous transmission from the on body device; and enabling communication circuitry of the reader device for a receiving period such that the communication circuitry is enabled at the expected time of receipt of the subsequent second synchronous transmission, where the receiving period is less than the length of the communication interval.

In some embodiments, the method further includes receiving the subsequent second transmission from the on body device and disabling the communication circuitry. In some embodiments, the method further includes: determining, by the reader device, an expected time of receipt of a subsequent third synchronous transmission from the on body device; and enabling communication circuitry of the reader device for a second receiving period such that the communication circuitry is enabled at the expected time of receipt of the subsequent third synchronous transmission, where the second receiving period is less than the length of the communication interval.

In some embodiments, the reader device uses power to monitor for the subsequent second synchronous transmission from the on body device during the receiving period. In some embodiments, the reader device does not use power to monitor for the subsequent second synchronous transmission from the on body device at times outside of the receiving period.

In many embodiments, a reader device for use in an analyte monitoring system is provided, the reader device adapted to communicate with an on body device at a communication interval, the reader device including: wireless communication circuitry; processing circuitry communicatively coupled with the wireless communication circuitry; and a non-transitory memory on which is stored a plurality of instructions that, when executed by the processing circuitry, cause the processing circuitry to: detect a loss of synchronization with the on body device; enable the wireless communication circuitry for a length of the communication interval to monitor for receipt of a first synchronous transmission from the on body device; disable the wireless communication circuitry after receipt of the first synchronous transmission; determine an expected time of receipt of a subsequent second synchronous transmission from the on body device; and enable the wireless communication circuitry of the reader device for a receiving period that includes the expected time of receipt of the subsequent second synchronous transmission, where the receiving period is less than the length of the communication interval.

In some embodiments, the plurality of instructions, when executed by the processing circuitry, cause the processing circuitry to: disable the wireless communication circuitry after receipt of the subsequent second synchronous transmission.

In some embodiments, the plurality of instructions, when executed by the processing circuitry, cause the processing circuitry to: determine an expected time of receipt of a subsequent third synchronous transmission from the on body device; and enable the wireless communication circuitry for a second receiving period that includes the expected time of receipt of the subsequent third synchronous transmission, where the second receiving period is less than the length of the communication interval.

In many embodiments, a method of reestablishing synchronization in an analyte monitoring system including an on body device and a reader device is provided, where synchronous transmissions occur at a communication interval, the method including: detecting a loss of synchronization at the reader device; performing a scan of the on body device, where the scan uses first communication circuitry of the reader device configured to communicate using inductive coupling; determining, by the reader device, an expected time of receipt of a synchronous transmission from the on body device, where the expected time of receipt is calculated from a time of the scan.

In some embodiments, detecting a loss of synchronization at the reader device includes: enabling second communication circuitry of the reader device for a receiving period less than a length of the communication interval; and failing to receive a synchronous communication from the on body device during the receiving period.

In some embodiments, the method further includes outputting a notification, by the reader device, for a user to perform the scan.

In some embodiments, performing a scan of the on body device includes: bringing the reader device within a distance of twelve inches from the on body device; transmitting a communication from the reader device to the on body device using inductive coupling; and receiving a response communication at the reader device from the on body device. A time of receipt of the response communication at the reader device can be used as a synchronization event by the reader device and where a time of transmission of the response communication by the on body device is used as a synchronization event by the on body device. The on body device can transmit the synchronous transmission at a time determined from the synchronization event.

In some embodiments, the method further includes enabling second communication circuitry of the reader device for a receiving period such that the second communication circuitry is enabled at the expected time of receipt of the synchronous transmission, where the receiving period is less than the length of the communication interval. The reader device can power the second communication circuitry to monitor for the synchronous transmission from the on body device during the receiving period. The reader device can abstain from powering the second communication circuitry to monitor for the synchronous transmission from the on body device at times outside of the receiving period.

In many embodiments, a reader device for use in an analyte monitoring system, the reader device adapted to communicate with an on body device at a communication interval is provided, the reader device including: first wireless communication circuitry configured to communicate using inductive coupling; second wireless communication circuitry configured to communicate synchronously; processing circuitry communicatively coupled with the first and second wireless communication circuitry; and a non-transitory memory on which is stored a plurality of instructions that, when executed by the processing circuitry, cause the processing circuitry to: detect a loss of synchronization with the on body device; cause performance of a scan of the on body device, where the scan uses the first communication circuitry of the reader device; determine an expected time of receipt of a synchronous transmission from the on body device, where the expected time of receipt is calculated from a time of the scan.

In some embodiments, the plurality of instructions, when executed by the processing circuitry, cause the processing circuitry to detect loss of synchronization by: failure to receive a synchronous communication from the on body device during enablement of the second wireless communication circuitry for a receiving period less than a length of the communication interval.

In some embodiments, the plurality of instructions, when executed by the processing circuitry, cause the processing circuitry to: cause output of a notification for a user to perform the scan.

In some embodiments, the plurality of instructions, when executed by the processing circuitry, cause the processing circuitry to: cause transmission of a communication from the reader device to the on body device using inductive coupling; and read a response communication received by the first wireless communication circuitry to complete the scan.

In some embodiments, the plurality of instructions, when executed by the processing circuitry, cause the processing circuitry to use a time of receipt of the response communication as a synchronization event.

In some embodiments, the plurality of instructions, when executed by the processing circuitry, cause the processing circuitry to enable the second wireless communication circuitry for a receiving period that includes the expected time of receipt of the synchronous transmission, where the receiving period is less than the length of the communication interval.

For each and every embodiment of a method disclosed herein, systems and devices capable of performing each of those embodiments are covered within the scope of the present disclosure. For example, embodiments of OBDs are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These OBD embodiments can be used and can be capable of use to implement those steps performed by a OBD from any and all of the methods described herein.

Computer program instructions for carrying out operations in accordance with the described subject matter can be stored on any non-transitory memory described herein and executed by processing circuitry communicatively coupled thereto. The computer program instructions can be written in any combination of one or more programming languages, including an object oriented programming language such as Java, JavaScript, Smalltalk, C++, C#, Transact-SQL, XML, PHP or the like and conventional procedural programming languages, such as the “C” programming language or similar programming languages. The program instructions may execute entirely on the user's computing device, partly on the user's computing device, as a stand-alone software package, partly on the user's computing device and partly on a remote computing device or entirely on the remote computing device or server. In the latter scenario, the remote computing device may be connected to the user's computing device through any type of network, including a local area network (LAN) or a wide area network (WAN), or the connection may be made to an external computer (for example, through the Internet using an Internet Service Provider).

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

As used herein and in the appended claims, the singular forms “a,” “an,” and “the” include plural referents unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-155. (canceled)

156. An analyte monitoring system, comprising:
(1) an on body device, comprising:
an analyte sensor, at least a portion of which is configured to be positioned under skin of a subject and in contact with a bodily fluid of the subject; and
sensor electronics disposed in an interior of the on body device and coupled with the analyte sensor, the sensor electronics comprising one or more processors coupled with memory, wherein the memory is configured to store instructions that, when executed by the one or more processors, cause the one or more processors to generate data representative of an in vivo analyte level of the subject;
(1) a reader device, comprising:
a first communication circuitry of the reader device configured to wirelessly communicate according to a radio frequency (RF) protocol;
a second communication circuitry of the reader device configured to wirelessly communicate according to a near field communication (NFC) protocol;
(3) a medication delivery device, comprising processing circuitry coupled with memory for storing instructions that, when executed by the processing circuitry, cause the processing circuitry to store a medication dosage amount,
wherein the reader device is configured to wirelessly receive the data representative of the in vivo analyte level of the subject from the on body device according to either or both of the RF protocol or the NFC protocol, and
wherein the reader device is further configured to wirelessly communicate with the medication delivery device according to either or both of the RF protocol or the NFC protocol.

157. The analyte monitoring system of claim 156, wherein the reader device comprises a smart phone.

158. The analyte monitoring system of claim 156, wherein the medication delivery device comprises an insulin pen.

159. The analyte monitoring system of claim 158, wherein the insulin pen comprises an injection needle, a cartridge configured to carry a drug, an interface configured to control an amount of the drug to be delivered, and an actuator configured to cause injection of the amount of the drug.

160. The analyte monitoring system of claim 156, wherein the reader device and the medication delivery device comprise an integrated device having a common housing.

161. The analyte monitoring system of claim 160, wherein the integrated device comprises a medication pump.

162. The analyte monitoring system of claim 160, wherein the integrated device comprises an insulin pen.

163. The analyte monitoring system of claim 156, wherein the on body device further comprises an adhesive patch configured to attach the on body device to the skin of the subject.

164. The analyte monitoring system of claim 156, wherein the RF protocol is a Bluetooth or Bluetooth Low Energy protocol.

165. The analyte monitoring system of claim 156, wherein the RF protocol is a formed in an ultra-high frequency (UHF) band.

166. The analyte monitoring system of claim 156, wherein the reader device further comprises a shared antenna coupled with the first communication circuitry and the second communication circuitry.

167. The analyte monitoring system of claim 156, wherein the reader device further comprises a first antenna coupled with the first communication circuitry and a second antenna coupled with the second communication circuitry.

168. The analyte monitoring system of claim 156, wherein the instructions stored in the memory of the medication delivery device, when executed by the processing circuitry, further cause the processing circuitry to control delivery of a medication.

169. The analyte monitoring system of claim 156, wherein the instructions stored in the memory of the medication delivery device, when executed by the processing circuitry, further cause the processing circuitry to calculate the medication dosage amount.

170. The analyte monitoring system of claim 156, wherein the instructions stored in the memory of the medication delivery device, when executed by the processing circuitry, further cause the processing circuitry to calculate a duration of a medication delivery.

171. The analyte monitoring system of claim 156, wherein the instructions stored in the memory of the medication delivery device, when executed by the processing circuitry, further cause the processing circuitry to calculate the medication dosage amount or a duration of a medication deliver based on the data representative of the in vivo analyte level of the subject.

172. The analyte monitoring system of claim 156, wherein the reader is further configured to wirelessly receive an indication of an alarm condition from the on body device according to the RF protocol.

173. The analyte monitoring system of claim 172, wherein the alarm condition comprises a predicted low analyte level condition.

174. The analyte monitoring system of claim 172, wherein the alarm condition comprises a predicted high analyte level condition.

175. The analyte monitoring system of claim 156, wherein the analyte sensor is a glucose sensor.
